(19) United States
(12) Patent Application Publication
(10) Pub. No.: US 2007/0004929 A1
(43) Pub. Date: Jan. 4, 2007

(54) Synthesis of labeled compounds

(76) Inventors: Rodolfo A. Martinez, Santa Fe, NM (US); Jurgen G. Schmidt, Los Alamos, NM (US); Marc A. Alvarez, Santa Fe, NM (US); Louis A. Silks, Los Alamos, NM (US)

Correspondence Address: LOS ALAMOS NATIONAL SECURITY, LLC, LOS ALAMOS NATIONAL LABORATORY, PPO. BOX 1663, LC/IP, MS A187, LOS ALAMOS, NM, 87545, US

(21) Appl. No.: 11/172,126

(22) Filed: Jun. 29, 2005

(51) Int. Cl. C07F 7/22; C07C 391/02; C07F 7/00

(52) U.S. Cl. 556/87; 534/11; 556/428; 562/899

(57) ABSTRACT

The present invention is directed to labeled compounds of the formula Ar-Z₁-Q-Z₂ where Ar is an aryl group is selected from the group consisting of 1-naphthyl, substituted 1-naphthyl, 2-naphthyl, substituted 2-naphthyl, and phenyl groups with the structure 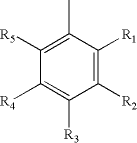
wherein R₁, R₂, R₃, R₄ and R₅ are each independently selected from the group consisting of hydrogen, a C₁-C₄ lower alkyl, a halogen, a phenyl, an alkoxy group and an amino group from the group consisting of NH₂, NHR and NRR′ where R and R′ are each a C₁-C₄ lower alkyl, Q is selected from the group consisting of ¹³CH₂, ¹³CDH and ¹³CD₂, and Z₁ is selected from the group consisting of —S—, —S(═O)—, —S(═O)₂—, —Se—, —Se(═O)—, and —Se(═O)₂—, Z₂ is selected from the group consisting of —Si(R₆R₇R₈), —O(R₉), —Se—Ar, —Se(═O)—Ar, —Se(═O)₂—Ar, —S—Ar, —S(═O)—Ar, and —S(═O)₂—Ar wherein R₆, R₇ and R₈ are each independently selected from the group consisting of a C₁-C₄ lower alkyl, R₉ is a C₁-C₄ lower alkyl or an R₁₀—Ar group where R₁₀ is a C₁-C₄ alkylene group.

### STATEMENT REGARDING FEDERAL RIGHTS

This invention was made with government support under Contract No. W-7405-ENG-36 awarded by the U.S. Department of Energy. The government has certain rights in the invention.

### FIELD OF THE INVENTION

The present invention relates to labeled compounds and more particularly to compounds labeled with carbon-13 and hydrogen-2.

### BACKGROUND OF THE INVENTION

Stable isotope labeled amino acids and nucleotides are required for structural and mechanistic studies of proteins and oligonucleotides. In addition, isotopically labeled biologically active compounds are required for many phases of drug discovery and development including elucidation of biosynthetic pathways, pharmacokinetics, and drug metabolism. For many applications, site-specific ¹³C or combined ¹³C and ²H labeling are required. While a number of stable isotope labeled compounds are available from companies such as Sigma-Aldrich Chemicals, a need remains for other labeled synthetic precursors.

Labeled dithianes have been previously developed (U.S. Pat. No. 6,541,671) and are useful for introduction of a carbon-13 and a hydrogen-2 or deuterium label into biochemicals and other precursor materials. Other labeled compounds such as methyl phenyl sulfone and methyl aryl sulfoxides such as methyl phenyl sulfoxide have also been developed (possible cite if other patent issues before filing). Availability of other significant labeled compounds would allow researchers to take advantage of the wealth of chemistry that has been done using similar unlabeled compounds.

As carbon-13 is separated from its lighter isotope by cyrogenic distillation of carbon monoxide (CO), all labeled carbons are derived ultimately from CO. The highly efficient conversion of CO to useful chemical precursors is perhaps the most unique aspect of stable isotope labeling technology. Any inefficiency in the early synthetic steps adds greatly to the overall expense of isotope labeling. Thus, considerable efforts have been directed to the development of methods for the preparation of useful synthetic precursors or synthons. This effort has given rise to efficient large-scale methods for the synthesis of methane, methanol, methyl iodide, sodium formate, potassium cyanide and carbon dioxide. These methods are the foundation of all labeling chemistry. The most useful of the electrophilic one-carbon precursors, methyl iodide and carbon dioxide, are difficult to store and use efficiently due to their high volatility.

As spectroscopic instrumentation and techniques continue to improve, there is a drive to study ever more complicated bio-systems. This has lead to demands for more complex labeling patterns in biomolecules. In the past, the simple introduction of a labeled atom site-specifically without stereospecificity was the major thrust for stable isotope labeling and the first generation of labeled synthons served this effort well. Increasingly, in today's labeling climate, in addition to site-specific labeling, the requirement for stereospecificity has been added. This includes both the ability to stereospecific label chiral compounds as well as the ability to differentiate between prochiral centers with deuterium or carbon. The development of additional synthons as starting materials will address those growing demands.

Accordingly, it is an object of the present invention to provide labeled compounds.

### SUMMARY OF THE INVENTION

In accordance with the purposes of the present invention, as embodied and broadly described herein, the present invention provides a labeled compound of the formula Ar-Z₁-Q-Z₂ where Ar is an aryl group is selected from the group consisting of 1-naphthyl, substituted 1-naphthyl, 2-naphthyl, substituted 2-naphthyl, and phenyl groups with the structure 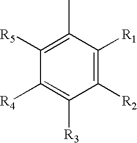
wherein R₁, R₂, R₃, R₄ and R₅ are each independently, hydrogen, a C₁-C₄ lower alkyl, a halogen, a phenyl, an alkoxy group and an amino group from the group consisting of NH₂, NHR and NRR′ where R and R′ are each a C₁-C₄ lower alkyl, Q is selected from the group consisting of ¹³CH₂, ¹³CDH and ¹³CD₂, Z₁ is selected from the group consisting of —S—, —S(═O)—, —S(═O)₂—, —Se—, —Se(═O)—, and —Se(═O)₂—, Z₂ is selected from the group consisting of —Si(R₆R₇R₈), —O(R₉), —Se—Ar, —Se(═O)—Ar, —Se(═O)₂Ar, —S—Ar, —S(═O)—Ar, and —S(═O)₂—Ar wherein R₆, R₇ and R₈ are each independently selected from the group consisting of a C₁-C₄ lower alkyl, R₉ is a C₁-C₄ lower alkyl or an R₁₀—Ar group where R₁₀ is a C₁-C₄ alkylene group.

Exemplary compounds of the present invention include trimethylsilyl-¹³C-methyl-phenylsulfide, trimethylsilyl-¹³C-methyl-phenylsulfone, tributylstannyl-¹³C-methyl-phenylsulfone, thiophenyl-¹³C-methyl-phenylselenide, tributylstannyl-¹³C-methyl-phenylsulfide.

The present invention further provides processes of preparing such labeled compounds.

### DETAILED DESCRIPTION

The present invention is concerned with isotopically labeled compounds and specifically isotopically labeled compounds including a carbon-13 [¹³C] and a hydrogen-2 or deuterium label [²H].

The present invention provides labeled compounds of the general formula Ar-Z₁-Q-Z₂ where Ar is an aryl group is from the group consisting of 1-naphthyl, substituted 1-naphthyl, 2-naphthyl, substituted 2-naphthyl, and phenyl groups with the structure 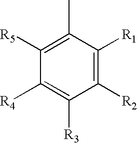
wherein R₁, R₂, R₃, R₄ and R₅ are each independently from the group of hydrogen, a C₁-C₄ lower alkyl, a halogen, a phenyl, an alkoxy group and an amino group from the group consisting of NH₂, NHR and NRR′ where R and R′ are each a C₁-C₄ lower alkyl, Q is selected from the group consisting of ¹³CH₂, ¹³CDH and ¹³CD₂, Z₁ is from the group consisting of —S—, —S(═O)—, —S(═O)₂—, —Se—, —Se(═O)—, and —Se(═O)₂—, Z₂ is from the group consisting of —Si(R₆R₇R₈), —O(R₉), —Se—Ar, —Se(═O)—Ar, —Se(═O)₂Ar, —S—Ar, —S(═O)—Ar, and —S(═O)₂—Ar wherein R₆, R₇ and R₈ are each independently from the group consisting of a C₁-C₄ lower alkyl, R₉ is a C₁-C₄ lower alkyl or an R₁₀—Ar group where R₁₀ is a C₁-C₄ alkylene group.

Among specific isotopically labeled compounds, the present invention is concerned with compounds including more than one atom from among sulfur, silicon, selenium and tin atoms such as trimethylsilyl-¹³C-methyl-phenylsulfide, trimethylsilyl-¹³C-methyl-phenylsulfone, tributylstannyl-¹³C-methyl-phenylsulfone, thiophenyl-¹³C-methyl-phenylselenide, tributylstannyl-¹³C-methyl-phenylsulfide and the like. Similar compounds can be prepared by using a suitable deuterated starting material to obtain the same compounds including one or two deuterium atoms on the ¹³C labeled methyl group. Such labeled compounds are useful organic reagents that allow for the preparation of many biochemicals and materials and can be used to introduce a carbon-13 [¹³C] and a hydrogen-2 or deuterium label [²H] into biochemicals and materials.

As used herein, the term “aryl” means a monovalent monocyclic or bicyclic aromatic hydrocarbon substituent of 6 to 10 ring atoms, and optionally substituted independently with one, two, three, four or five substituents selected from alkyl, haloalkyl, cycloalkyl, halo, nitro, cyano, —OR (where R is hydrogen, alkyl, haloalkyl, cycloalkyl, optionally substituted phenyl), acyl, and —COOR (where R is hydrogen or alkyl). More specifically, the term “aryl” includes, but is not limited to 1-naphthyl, substituted 1-naphthyl, 2-naphthyl, substituted 2-naphthyl, and phenyl groups with the structure 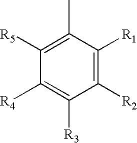
wherein R₁, R₂, R₃, R₄ and R₅ are each independently a lower alkyl, i.e., a C₁-C₄ alkyl such as methyl, ethyl, n-propyl, iso-propyl, butyl, isobutyl, and tert-butyl, a halogen such as chloro, bromo or iodo, an amino group such as NH₂, NHR or NRR′ where R and R′ are each a lower alkyl or aryl as described above, or an alkoxy group such as O-alkyl or O-aryl where the alkyl is a lower alkyl as described above or an aryl as described above.

As used herein, the terms “[¹³C, d] or [²H₁, ¹³C]” mean exactly one deuterium atom within a respective compound, the terms “[¹³C, d₂] or [²H₂, ¹³C]” mean exactly two deuterium atoms within a respective compound and the terms “[¹³C, d₃] or [²H₃, ¹³C]” mean exactly three deuterium atoms within a respective compound.

The present invention uses known labeled compounds including [¹³C], [²H₁, ¹³C], [²H₂, ¹³C] or [²H₃, ¹³C] such as described in U.S. Pat. Nos. 6,713,044 and 6,764,673. Such labeled compounds wherein, e.g., the ¹³C methyl group, includes exactly one, two or three deuterium atoms, attached to a sulfur group or other suitable group can be used in the synthesis of the deuterium labeled compounds of the present invention.

The present invention is more particularly described in the following examples which are intended as illustrative only, since numerous modifications and variations will be apparent to those skilled in the art.

The starting materials of [¹³C]methyl phenyl sulfide and [¹³C, d₃]methyl phenyl sulfide were prepared in accordance with the preparation described in U.S. Pat. No. 6,713,044 by Martinez et al. such description incorporated herein by reference.

### EXAMPLE 1

Trimethylsilyl-¹³C-methyl-phenylsulfide[C₆H₅S¹³CH₂ TMS] was prepared as follows. ¹³C-methyl-phenylsulfide (250 milligrams (mg); 2 millimoles (mmol)) was dissolved in dry tetrahydrofuran (THF) (30 mL) in a flame dried flask under argon and cooled to −78° C. in a dry ice bath. A solution of sec-butyllithium (sec-BuLi) in cyclohexane (1.7 mL; 2.1 mmol; 1.24 M solution) was added dropwise via syringe, and the solution was stirred at −78° C. for 1.5 hours. Then, chlorotrimethylsilane (TMSCl) (266 microliters (μl); 2.1 mmol) was added and the reaction mixture was stirred for 30 minutes at −78° C., then warmed to −20° C. over about 2 hours and quenched at this temperature by the addition of a saturated solution of ammonium chloride (20 mL). The phases were separated, the water phase extracted three times with Ether (50 mL) and the combined organic phases washed three times with brine (NaCl solution) (30 mL). After drying (over sodium sulfate), filtration and evaporation, the remaining oily residue was chromatographed (FLC; gradient Hexanes to Hexanes/Ether 1:1) on silica gel to yield trimethylsilyl-¹³C-methyl-phenylsulfide (365 mg; 1.84 mmol; 92% yield, a corrected value based on NMR integration) as a colorless oil. This oil contained about 1% BisTMS-¹³C-methyl-phenylsulfide and less than 1% starting material, such impurities were inseparable by FLC from the major product.

Further purification can be accomplished by distillation upon scaleup. Also, alternative reagents and reaction conditions may improve yields, e.g., TMSOMe (trimethylsilyl)methanol) and MSTFA (N-methyl-N-(trimethylsilyl)trifluoroacetamide), or ¹³C-methyl-phenylsulfoxide with trifluoromethylsulfonate (TMSOTf) and HMDS (hexamethyldisilazane).

### EXAMPLE 2

Trimethylsilyl-¹³C-methyl-phenylsulfone [C₆H₅SO₂¹³CH₂ TMS] was prepared as follows. ¹³C-methyl-phenylsulfone (500 mg; 3.18 mmol) was dissolved in dry THF (30 mL) in a flame dried flask under argon and cooled to −78° C. in a dry ice bath. A solution of sec-BuLi in Hexanes (1.46 mL; 3.5 mmol; 2.4 M solution) was added dropwise, and the solution was stirred for 30 minutes at −78° C. Then, chlorotrimethylsilane (423 μl; 3.34 mmol) was added and the reaction mixture was stirred for 2 hours at −78° C., then warmed to −20° C. over about 3 hours and quenched at this temperature by the addition of a saturated solution of ammonium chloride (20 mL). The phases were separated, the water phase extracted twice with Ether (50 mL) and the combined organic phases washed three times with brine (30 mL). After drying (over sodium sulfate), filtration and evaporation, the remaining yellowish oily residue was chromatographed (FLC; gradient Hexanes/Ether 2:1 to 1:1) on silica gel to yield trimethylsilyl-¹³C-methyl-phenylsulfone (631 mg; 2.75 mmol; 86.5% yield) as white, wax-like crystals.

Improved yield may be obtained by deprotanation with LiHMDS (lithium hexamethyldisilazane) and reaction with chlorotrimethylsilane may give better results in terms of yield and side-product formation.

### EXAMPLE 3

Tributylstannyl-¹³C-methyl-phenylsulfone [C₆H₅SO₂¹³CH₂Sn(n-Bu)₃] was prepared as follows. ¹³C-methyl-phenylsulfone (500 mg; 3.18 mmol) was dissolved in dry THF (30 mL) in a flame dried flask under argon and cooled to −78° C. in a dry ice bath. A solution of sec-BuLi in Hexanes (1.46 mL; 3.5 mmol; 2.4 M solution) was added dropwise, and the solution was stirred for 30 minutes at −78° C. Then, n-Tributyltinchloride (905 μl; 3.34 mmol) was added and the reaction mixture was stirred for 2 hours at −78° C., then warmed to −20° C. over about 1 hour and quenched at this temperature by the addition of a saturated solution of ammonium chloride (20 mL). The phases were separated, the water phase extracted twice with Ether (50 mL) and the combined organic phases washed three times with brine (30 mL). After drying (over sodium sulfate), filtration and evaporation, the remaining yellowish oily residue was chromatographed (FLC; gradient Hexanes/Et₂O 2:1) on silica gel to yield tributylstannyl-¹³C-methyl-phenylsulfone (1.264 g; 2.83 mmol; 89.1% yield) as a colorless oil.

Improved yield may be obtained by reaction of the starting material with Bu₃SnH, base and palladium catalysts.

### EXAMPLE 4

Tributylstannyl-¹³C-methyl-phenylsulfide [C₆H₅S¹³CH₂Sn(n-Bu)₃] was prepared as follows. ¹³C-methyl-phenylsulfide (2.5 g; 20 mmol) was dissolved in dry THF (30 mL) in a flame dried flask under argon and cooled to −78° C. in a dry ice bath. A solution of sec-BuLi in cyclohexane (16.5 mL; 20 mmol; 1.21 M solution) was added dropwise, and the solution was stirred for 2 hours at −78° C. Then, n-Tributyltinchloride (5.32 mL; 19.6 mmol) was added and the reaction mixture was stirred for 3 hours at −78° C., then warmed to 0° C. over about 5 hours and quenched at this temperature by the addition of a saturated solution of sodium chloride (100 mL). Ether (250 mL) was added, the phases were separated, the water phase extracted twice with Ether (100 mL) and the combined organic phases washed three times with brine (100 mL). After drying (over sodium sulfate), filtration and evaporation, the remaining yellowish oily residue was chromatographed (FLC; Hexanes) on silica gel to yield tributylstannyl-¹³C-methyl-phenylsulfide (7.852 g; 18.96 mmol; 96.7% yield) as a colorless oil, together with 1.3% of the starting material of ¹³C-methyl-phenylsulfide (32.8 mg; 0.262 mmol).

The reaction was conducted with n-tributyltinchloride as the limiting reagent in order to avoid a complicated separation of the product from unreacted tin compound. Thus, the reaction yield was calculated on the amount of the n-Tributyltinchloride and not the labeled compound.

### EXAMPLE 5

Thiophenyl-¹³C-methyl-phenylselenide [C₆H₅Se¹³CH₂SC₆H₅] was prepared as follows. ¹³C-methyl-phenylsulfide (125.2 mg; 1 mmol) was dissolved in dry THF (50 mL) in a flame dried flask under argon and cooled to −78° C. in a dry ice bath. A solution of sec-BuLi in cyclohexane (0.85 mL; 1 mmol; 1.19 M solution) was added dropwise, and the solution was stirred for 2 hours at −78° C. Then, diphenyldiselenide (312 mg; 1 mmol) was added and the reaction mixture was stirred for 1 hour at −78° C., then warmed to 0° C. over about 4 hours and quenched at this temperature by the addition of 1N HCl (5 mL). The phases were separated, the water phase extracted twice with ethylacetate (50 mL) and the combined organic phases washed three times with brine (30 mL). After drying (over sodium sulfate), filtration and evaporation, the remaining yellowish oily residue was chromatographed (FLC; Hexanes/methlyene chloride 2:1) on silica gel to yield thiophenyl-¹³C-methyl-phenylselenide (251 mg; 0.895 mmol; 89.5% yield) as a slightly yellow oil, and recovery of 5.6 mg of the ¹³C-methyl-phenylsulfide starting material.

The reaction yield based on consumed starting material was 90.8%. A similar reaction run with PhSeCl rather than the (PhSe)₂ gave a lower yield of 51.7% and recovery of starting material. The reaction yield of this PhSeCl reaction based on recovered starting material was 88% and this reaction yield may be increased with use of freshly distilled PhSeCl.

Although the present invention has been described with reference to specific details, it is not intended that such details should be regarded as limitations upon the scope of the invention, except as and to the extent that they are included in the accompanying claims.

What is claimed is:

1. A labeled compound of the formula Ar-Z₁-Q-Z₂ where Ar is an aryl group is selected from the group consisting of 1-naphthyl, substituted 1-naphthyl, 2-naphthyl, substituted 2-naphthyl, and phenyl groups with the structure 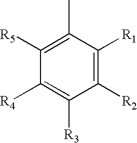
wherein R₁, R₂, R₃, R₄ and R₅ are each independently selected from the group consisting of hydrogen, a C₁-C₄ lower alkyl, a halogen, a phenyl, an alkoxy group and an amino group from the group consisting of NH₂, NHR and NRR′ where R and R′ are each a C₁-C₄ lower alkyl, Q is selected from the group consisting of ¹³CH₂, ¹³CDH and ¹³CD₂, and Z₁ is selected from the group consisting of —S—, —S(═O)—, —S(═O)₂—, —Se—, —Se(═O)—, and —Se(═O)₂—, Z₂ is selected from the group consisting of —Si(R₆R₇R₈), —O(R₉), —Se—Ar, —Se(═O)—Ar, —Se(═O)₂—Ar, —S—Ar, —S(═O)—Ar, and —S(═O)₂—Ar wherein R₆, R₇ and R₈ are each independently selected from the group consisting of a C₁-C₄ lower alkyl, R₉ is a C₁-C₄ lower alkyl or an R₁₀—Ar group where R₁₀ is a C₁-C₄ alkylene group.

2. The labeled compound of claim 1 wherein Ar is phenyl, R₆, R₇ and R₈ are each methyl and R₁₀ is methylene.

3. The labeled compound of claim 1 wherein said compound is trimethylsilyl-¹³C-methyl-phenylsulfide.

4. The labeled compound of claim 1 wherein said compound is trimethylsilyl-¹³C, d₂-methyl-phenylsulfide.

5. The labeled compound of claim 1 wherein said compound is trimethylsilyl-¹³C, d₁-methyl-phenylsulfide.

6. The labeled compound of claim 1 wherein said compound is trimethylsilyl-¹³C-methyl-phenylsulfone.

7. The labeled compound of claim 1 wherein said compound is trimethylsilyl-¹³C,d₂-methyl-phenylsulfone.

8. The labeled compound of claim 1 wherein said compound is trimethylsilyl-¹³C,d₁-methyl-phenylsulfone.

9. The labeled compound of claim 1 wherein said compound is thiophenyl-¹³C-methyl-phenylselenide.

10. The labeled compound of claim 1 wherein said compound is thiophenyl-¹³C,d₂-methyl-phenylselenide.

11. The labeled compound of claim 1 wherein said compound is thiophenyl-¹³C,d₁-methyl-phenylselenide.

12. The labeled compound of claim 1 wherein said compound is tributylstannyl-¹³C-methyl-phenylsulfone.

13. The labeled compound of claim 1 wherein said compound is tributylstannyl-¹³C,d₂-methyl-phenylsulfone.

14. The labeled compound of claim 1 wherein said compound is tributylstannyl-¹³C,d₁-methyl-phenylsulfone.

15. The labeled compound of claim 1 wherein said compound is tributylstannyl-¹³C-methyl-phenylsulfide.

16. The labeled compound of claim 1 wherein said compound is tributylstannyl-¹³C,d₂-methyl-phenylsulfide.

17. The labeled compound of claim 1 wherein said compound is tributylstannyl-¹³C,d₁-methyl-phenylsulfide.

18. The labeled compound of claim 1 wherein said compound is thiophenyl-¹³C-methyl-phenylsulfide.
